(19) United States
(12) Patent Application Publication
(10) Pub. No.: US 2019/0200897 A1
(43) Pub. Date: Jul. 4, 2019

(54) MICRO ACETONE DETECTING DEVICE

(71) Applicant: Microjet Technology Co., Ltd., Hsinchu (TW)

(72) Inventors: Hao-Jan MOU, Hsinchu (TW); Hung-Hsin LIAO, Hsinchu (TW); Shih-Chang CHEN, Hsinchu (TW); Jia-Yu LIAO, Hsinchu (TW); Shou-Hung CHEN, Hsinchu (TW); Chi-Feng HUANG, Hsinchu (TW); Yung-Lung HAN, Hsinchu (TW); Wei-Ming LEE, Hsinchu (TW)

(73) Assignee: Microjet Technology Co., Ltd., Hsinchu (TW)

(21) Appl. No.: 16/202,778

(22) Filed: Nov. 28, 2018

(30) Foreign Application Priority Data
Dec. 29, 2017 (TW) 106146541

(51) Int. Cl. A61B 5/08; G01N 33/497; A61B 5/097

(52) U.S. Cl. CPC A61B 5/082; A61B 5/0002; A61B 5/097; G01N 33/497

(57) ABSTRACT

A micro acetone detecting device is provided. The micro acetone detecting device includes a circuit board, a casing, an acetone sensor and an air pump. The casing has a first through hole and a second through hole. The casing is assembled on the circuit board, and an interior of the casing and the circuit board define an accommodation space. The acetone sensor is disposed in the accommodation space and electrically connected to the circuit board. The air pump is disposed in the accommodation space and electrically connected to the circuit board. When the air pump is actuated to change the pressure of the gas in the accommodation space, the gas flows into the accommodation space through the first through hole, and the gas in the accommodation space is measured by the acetone sensor to obtain an acetone concentration, and then the gas is discharged out through the second through hole.

### FIELD OF THE INVENTION

The present disclosure relates to an acetone detecting device, and more particularly to a micro acetone detecting device, which utilizes an air pump to enhance the efficiency of detecting acetone and calculates blood glucose concentration according to the acetone concentration.

### BACKGROUND OF THE INVENTION

For diabetes mellitus patients, self-detection of blood glucose plays an important role in the management of blood glucose. Currently, the blood glucose meter used to measure the blood glucose is inconvenient to carry around, so it is difficult for patients to monitor the blood glucose level when they go out. In addition, in the process of measuring the blood glucose, sometimes there is no bleeding or too little blood is drawn when a needle is employed to draw the blood. Hence, it is necessary to use the needle again or force to squeeze the blood out. This may cause the psychological fear of the patient, and forcing to squeeze the blood out may result in incorrect measuring results.

Therefore, there is a need of providing a micro acetone detecting device to address the above-mentioned issues encountered by the prior arts. The micro acetone detecting device should be safe to use and convenient to carry around. The micro acetone detecting device may calculate the blood glucose concentration according to the acetone concentration in the gas exhaled by the user, measure the blood glucose concentration of the user by using painless and convenient manners, and allow the patients to measure the blood glucose concentration in daily life easily and at any time.

### SUMMARY OF THE INVENTION

The object of the present disclosure is to provide a micro acetone detecting device to measure the blood glucose concentration effectively and conveniently. In accordance with an aspect of the present disclosure, a micro acetone detecting device is provided. The micro acetone detecting device includes a circuit board, a casing, an acetone sensor and an air pump. The casing has a first through hole and a second through hole. The casing is assembled on the circuit board, and an interior of the casing and the circuit board define an accommodation space. The acetone sensor is disposed in the accommodation space and electrically connected to the circuit board. The air pump is disposed in the accommodation space and electrically connected to the circuit board. When the air pump is actuated to change the pressure of gas in the accommodation space, the gas flows into the accommodation space through the first through hole, and the gas in the accommodation space is measured by the acetone sensor to obtain an acetone concentration, and then the gas is discharged out through the second through hole.

The above contents of the present disclosure will become more readily apparent to those ordinarily skilled in the art after reviewing the following detailed description and accompanying drawings, in which:

### BRIEF DESCRIPTION OF THE DRAWINGS

FIG. 1 is a schematic cross-sectional view illustrating a micro acetone detecting device according to a first embodiment of the present disclosure, wherein the arrow-shaped symbols indicate the direction for guiding gas;

FIG. 2 is a schematic cross-sectional view illustrating the micro acetone detecting device according to a second embodiment of the present disclosure, wherein the arrow-shaped symbols indicate the direction for guiding the gas;

FIG. 3 is a schematic cross-sectional view illustrating the micro acetone detecting device according to a third embodiment of the present disclosure, wherein the arrow-shaped symbols indicate the direction for guiding the gas;

FIG. 4 is a schematic cross-sectional view illustrating the micro acetone detecting device according to a fourth embodiment of the present disclosure, wherein the arrow-shaped symbols indicate the direction for guiding the gas;

FIG. 5 is a schematic cross-sectional view illustrating the micro acetone detecting device according to a fifth embodiment of the present disclosure, wherein the arrow-shaped symbols indicate the direction for guiding the gas;

FIG. 6 is a schematic cross-sectional view illustrating the micro acetone detecting device according to a sixth embodiment of the present disclosure, wherein the arrow-shaped symbols indicate the direction for guiding the gas;

FIG. 7 is a schematic exploded view illustrating an air pump of the micro acetone detecting device according to the third and fourth embodiments of the present disclosure;

FIG. 8 is a schematic perspective view illustrating the air pump disposed in a casing of the micro acetone detecting device according to the third embodiment of the present disclosure; and

FIG. 9 is a schematic exploded view illustrating the air pump of the micro acetone detecting device according to the fifth and sixth embodiments of the present disclosure.

### DETAILED DESCRIPTION OF THE PREFERRED EMBODIMENT

The present disclosure will now be described more specifically with reference to the following embodiments. It is to be noted that the following descriptions of preferred embodiments of this disclosure are presented herein for purpose of illustration and description only. It is not intended to be exhaustive or to be limited to the precise form disclosed.

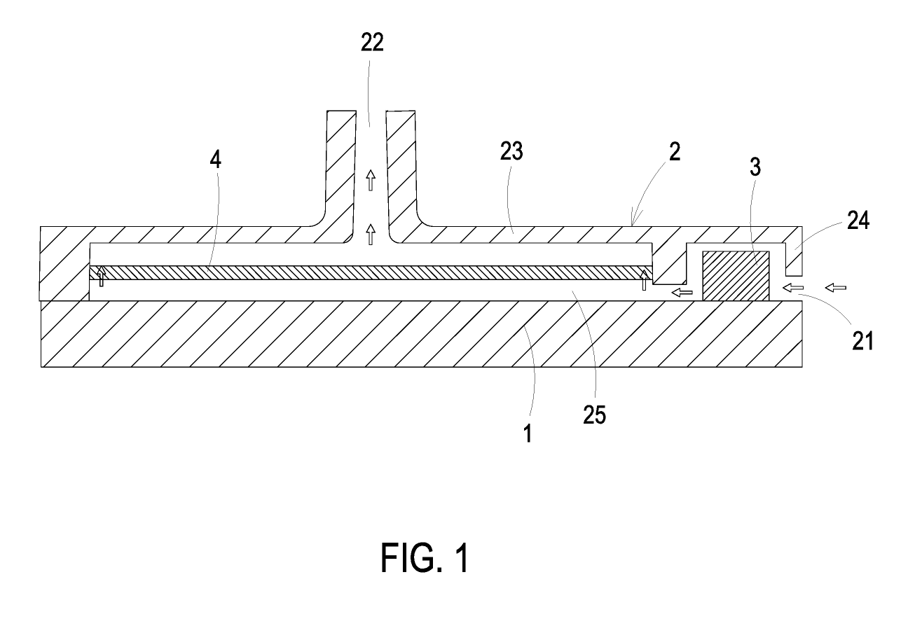

Please refer to FIG. 1. The present discourse provides a micro acetone detecting device including at least one circuit board 1, at least one casing 2, at least one first through hole 21, at least one second through hole 22, at least one accommodation space 25, at least one acetone sensor 3 and at least one air pump 4. The number of the circuit board 1, the casing 2, the first through hole 21, the second through hole 22, the accommodation space 25, the acetone sensor 3 and the air pump 4 is exemplified by one for each in the following embodiments but not limited thereto. It is noted that the circuit board 1, the casing 2, the first through hole 21, the second through hole 22, the accommodation space 25, the acetone sensor 3 and the air pump 4 can also be provided in plural numbers.

Please refer to FIG. 1, which is a schematic cross-sectional view illustrating a micro acetone detecting device according to a first embodiment of the present disclosure, wherein the arrow-shaped symbols indicate the direction for guiding gas. The micro acetone detecting device includes a circuit board 1, a casing 2, an acetone sensor 3 and an air pump 4. The casing 2 has a first through hole 21 and a second through hole 22, and the casing 2 includes a bottom plate 23 and a sidewall 24. The sidewall 24 extends from and is perpendicular to the periphery of the bottom plate 23. The casing 2 is assembled on the circuit board 1. A region enclosed by the bottom plate 23, the sidewall 24 and the circuit board 1 defines an accommodation space 25. The accommodation space 25 is in fluid communication with the first through hole 21 and the second through hole 22. The acetone sensor 3 is disposed in the accommodation space 25 and electrically connected to the circuit board 1. The air pump 4 is disposed in the accommodation space 25 and electrically connected to the circuit board 1. When the air pump 4 is actuated, the pressure of gas in the accommodation space 25 is changed. In this embodiment, the first through hole 21 is formed in the sidewall 24 of the casing 2 for introducing the gas therein. The second through hole 22 is formed in the bottom plate 23 of the casing 2 for discharging the gas thereout. The acetone sensor 3 is disposed adjacent to the first through hole 21, which is used for introducing the gas therein. When the air pump 4 is actuated, the gas is introduced through the first through hole 21 into the accommodation space 25, and the acetone concentration of the gas in the accommodation space 25 is measured by the acetone sensor 3 in the accommodation space 25 so that the acetone concentration of the gas can be measured at the first moment as the gas flows in. Finally, the gas is discharged out through the second through hole 22.

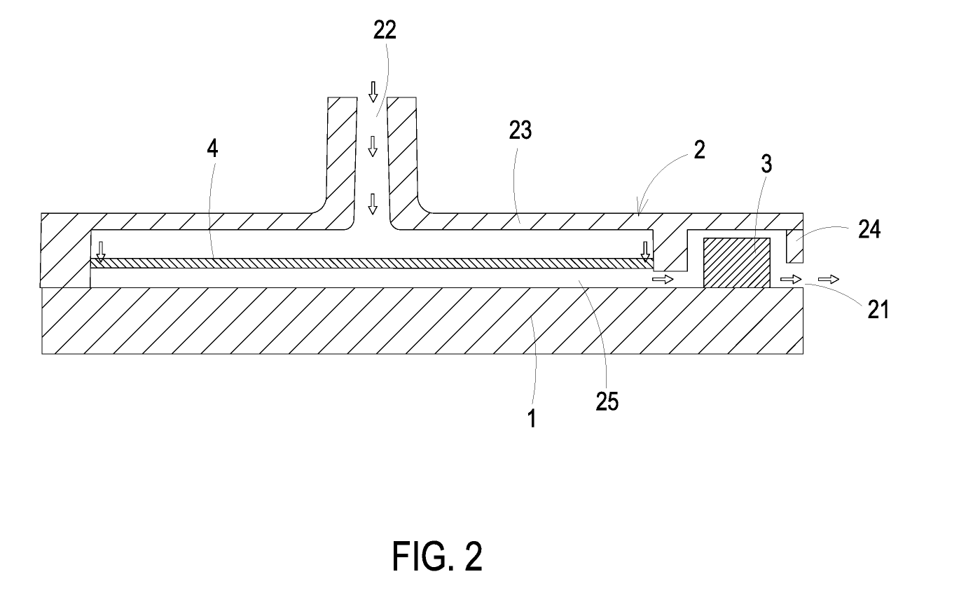

Please refer to FIG. 2, which is a schematic cross-sectional view illustrating the micro acetone detecting device according to a second embodiment of the present disclosure, wherein the arrow-shaped symbols indicate the direction for guiding the gas. In this embodiment, component parts and elements corresponding to those of the first embodiment are designated by identical numeral references, and detailed descriptions thereof are omitted. The gas guiding action is distinguished and described as follows. In this embodiment, the second through hole 22 is formed in the bottom plate 23 of the casing 2 for introducing the gas therein. The first through hole 21 is formed in the sidewall 24 of the casing 2 for discharging the gas thereout. The acetone sensor 3 is disposed adjacent to the first through hole 21, which is used for discharging the gas thereout. As the air pump 4 is actuated to compress and converge the gas, the acetone sensor 3 is provided with the gas for measuring.

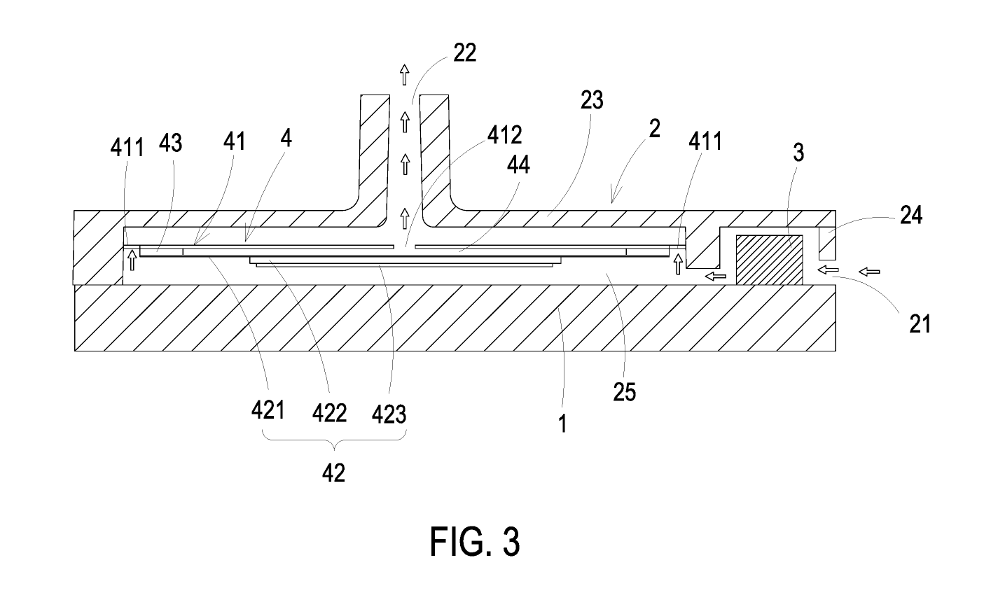

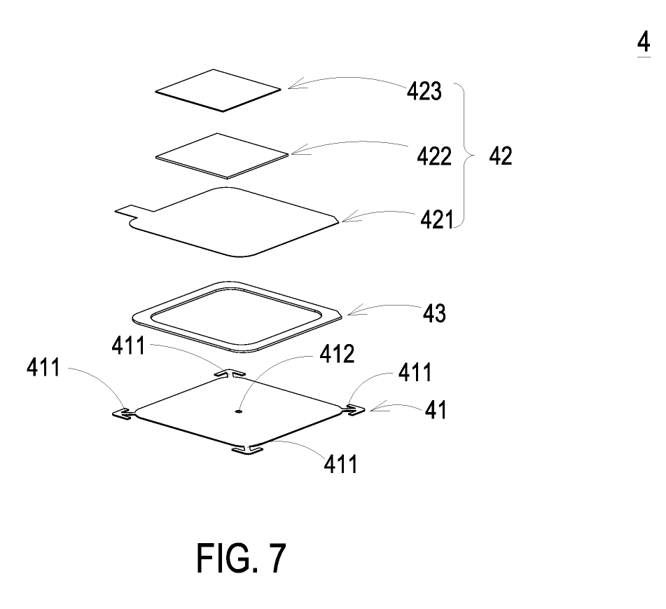

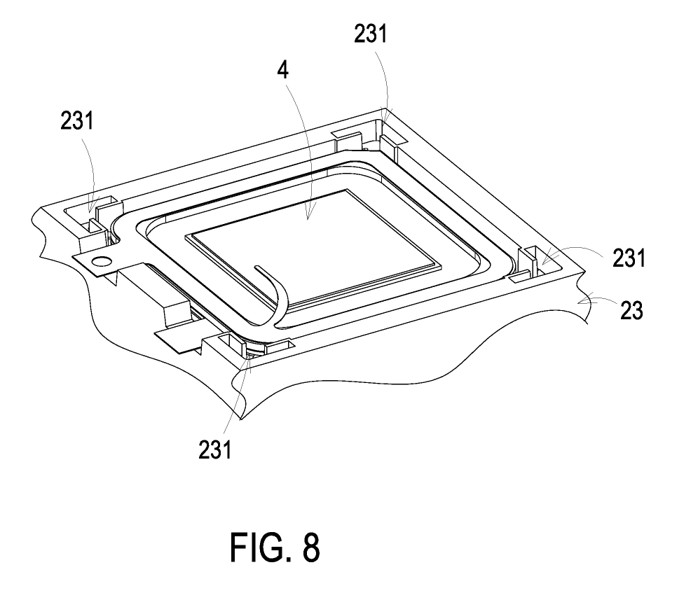

Please refer to FIGS. 3, 7 and 8. FIG. 3 is a schematic cross-sectional view illustrating the micro acetone detecting device according to a third embodiment of the present disclosure, wherein the arrow-shaped symbols indicate the direction for guiding the gas. FIG. 7 is a schematic exploded view illustrating an air pump of the micro acetone detecting device according to the third and fourth embodiments of the present disclosure. FIG. 8 is a schematic perspective view illustrating the air pump disposed in a casing of the micro acetone detecting device according to the third embodiment of the present disclosure. The third embodiment is a derivative embodiment of the first embodiment. The detailed structures and actions of the air pump 4 are described as follows. Component parts and elements corresponding to those of the first embodiment are designated by identical numeral references, and detailed descriptions thereof are omitted. As shown in FIG. 7, the air pump 4 includes a nozzle plate 41 and a piezoelectric assembly 42, which are stacked with each other. A frame 43 is disposed between the nozzle plate 41 and the piezoelectric assembly 42 so that the nozzle plate 41 and the piezoelectric assembly 42 are spaced apart from each other. The piezoelectric assembly 42, the frame 43 and the nozzle plate 41 are stacked sequentially from bottom to top and assembled together, and thus the nozzle plate 41 faces the bottom plate 23. The nozzle plate 41 includes a plurality of supporting parts 411 and a central aperture 412. The nozzle plate 41 is constructed on the casing 2 by the supporting parts 411, so that the nozzle plate 41 and the bottom plate 23 of the casing 2 are spaced apart from each other. In this embodiment, as shown in FIG. 8, the bottom plate 23 of the casing 2 has a plurality of fixing recesses 231, and the supporting parts 411 are disposed and positioned in the fixing recesses 231 of the bottom plate 23, so that the nozzle plate 41 and the bottom plate 23 are spaced apart from each other. A plurality of vacant spaces are defined between the supporting parts 411 for the gas flowing therethrough. The piezoelectric assembly 42 includes a piezoelectric plate 423, an auxiliary plate 422 and a vibration plate 421, which are stacked sequentially from top to bottom, and thus the piezoelectric plate 423 faces the circuit board 1. The auxiliary plate 422 is disposed between the piezoelectric plate 423 and the vibration plate 421, and served as a buffer between the piezoelectric plate 423 and the resonance plate 421 so that the vibration frequency of the vibration plate 421 can be adjusted. Basically, the thickness of the auxiliary plate 422 is larger than the thickness of the vibration plate 421, and the thickness of the auxiliary plate 422 may be designed so as to adjust the vibration frequency of the piezoelectric assembly 42. A region enclosed by the nozzle plate 41, the frame 43 and the piezoelectric assembly 42 defines a resonance chamber 44. By controlling the vibration frequency of the gas in the resonance chamber 44 to be close or even the same as the vibration frequency of the nozzle plate 41, the Helmholtz resonance effect can be generated between the resonance chamber 44 and the nozzle plate 41 for allowing the gas to be discharged out through the central aperture 412 of the nozzle plate 41. Consequently, air transportation efficiency is enhanced. As a voltage is applied to the piezoelectric plate 423 of the air pump 4, the piezoelectric plate 423 deforms owing to the piezoelectric effect. The piezoelectric plate 423 drives the piezoelectric assembly 42 and the nozzle plate 41 to vibrate up and down, thereby changing the pressure of the gas in the accommodation space 25. The environmental gas outside the micro acetone detecting device is inhaled into the accommodation space 25 through the first through hole 21, and the acetone sensor 3 disposed adjacent to the first through hole 21 can measure the gas flowing in through the first through hole 21 and obtain the acetone concentration of the gas in real time. Then, the gas flows through the vacant spaces among the supporting parts 411 and discharged out through the second through hole 22. By discharging the gas in the resonance chamber 44 through the central aperture 412 of the nozzle plate 41, the transportation efficiency of discharging the gas through the second through hole 22 can be enhanced. The piezoelectric assembly 42 and the nozzle plate 41 are driven to vibrate up and down repeatedly, so that the gas can be inhaled into the accommodation space 25 and discharged out from the accommodation space 25 into the environment, thereby allowing the acetone sensor 3 to measure the acetone concentration of the gas continuously.

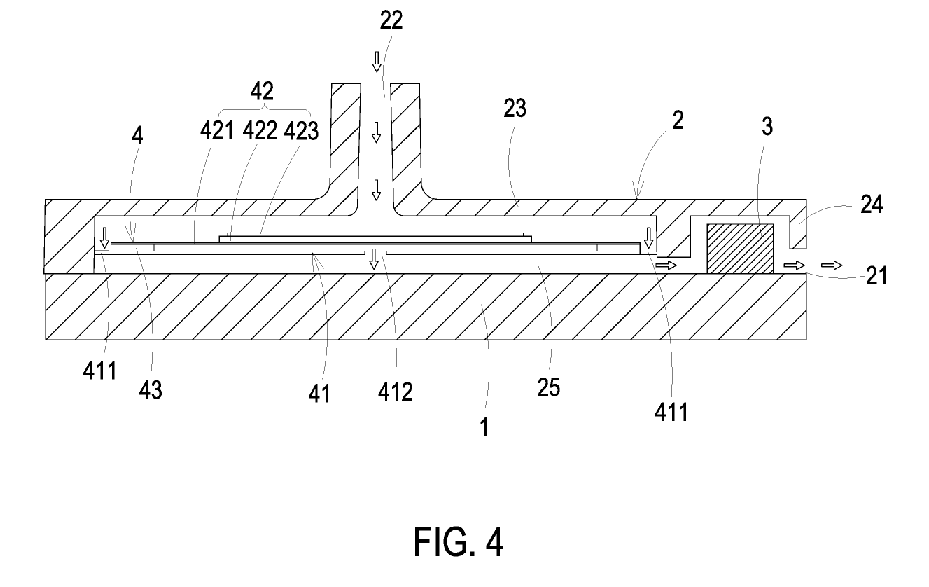

Please refer to FIG. 4, which is a schematic cross-sectional view illustrating the micro acetone detecting device according to a fourth embodiment of the present disclosure, wherein the arrow-shaped symbols indicate the direction for guiding the gas. The fourth embodiment is a derivative embodiment of the second embodiment. The detailed structures and actions of the air pump 4 are described as follows. Component parts and elements corresponding to those of the second embodiment are designated by identical numeral references, the structures of the air pump 4 are the same as that of the third embodiment, and detailed descriptions thereof are omitted. In this embodiment, the air pump 4 is constructed on the casing 2 reversely by the supporting parts 411 so that the air pump 4 and the bottom plate 23 of the casing 2 are spaced apart from each other. That is, in the assembly of the air pump 4 and the casing 2, the piezoelectric assembly 42 faces the bottom plate 23 of the casing 2. More specifically, the nozzle plate 41 in the third embodiment faces the bottom plate 23 of the casing 2, but the nozzle plate 41 in the fourth embodiment faces the circuit board 1. The direction of assembling the air pump 4 with the bottom plate 23 of the casing 2 in the fourth embodiment is reverse to the direction of assembling the air pump 4 with the bottom plate 23 of the casing 2 in the third embodiment. As a voltage is applied to the piezoelectric plate 423 of the air pump 4, the piezoelectric plate 423 deforms owing to the piezoelectric effect. The piezoelectric plate 423 drives the piezoelectric assembly 42 and the nozzle plate 41 to vibrate up and down, thereby changing the pressure of gas in the accommodation space 25. The environmental gas outside the micro acetone detecting device is inhaled into the accommodation space 25 through the second through hole 22, and the gas flows through the vacant spaces among the supporting parts 411. By discharging the gas in the resonance chamber 44 through the central aperture 412 of the nozzle plate 41, the transportation efficiency of discharging the gas through the first through hole 21 can be enhanced, and the gas is accelerated to guide to the acetone sensor 3 adjacent to the first through hole 21. Consequently, the acetone sensor 3 can measure the gas inhaled by the air pump 4 rapidly and obtain the acetone concentration of the gas in real time. The piezoelectric assembly 42 and the nozzle plate 41 are driven to vibrate up and down repeatedly so that the gas can be inhaled into the accommodation space 25 to be compressed and discharged out from the accommodation space 25 into the environment so that the acetone sensor 3 can measure the acetone concentration of the gas continuously.

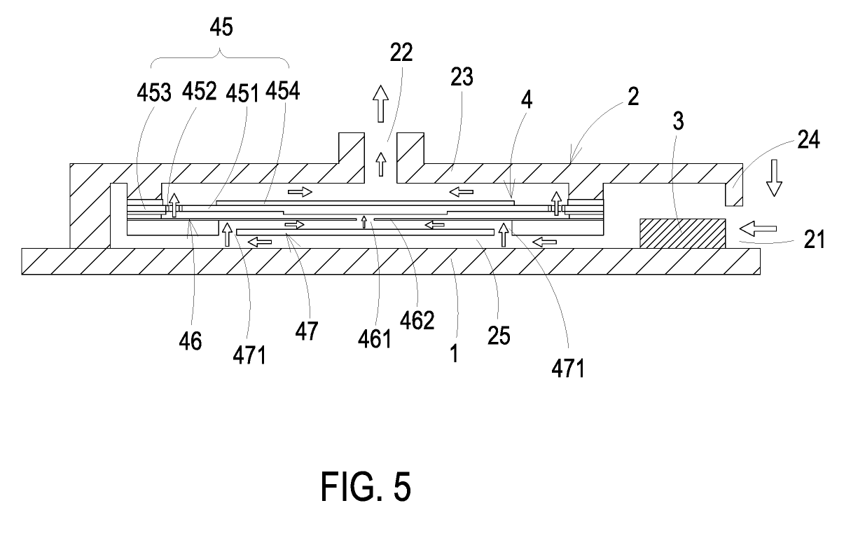

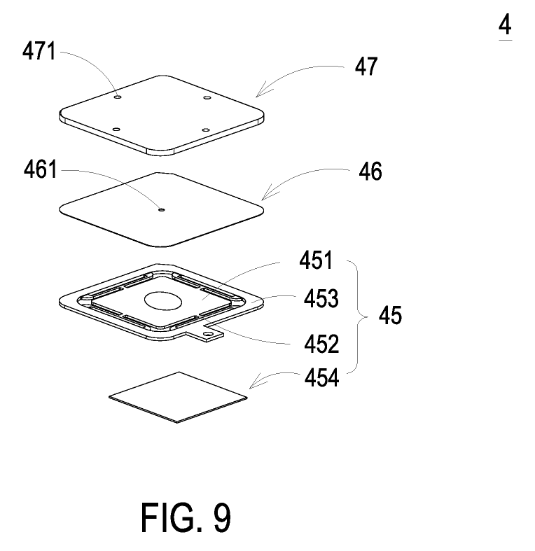

Please refer to FIGS. 5 and 9. FIG. 5 is a schematic cross-sectional view illustrating the micro acetone detecting device according to a fifth embodiment of the present disclosure, wherein the arrow-shaped symbols indicate the direction for guiding the gas. FIG. 9 is a schematic exploded view illustrating the air pump of the micro acetone detecting device according to the fifth and sixth embodiments of the present disclosure. The fifth embodiment is a derivative embodiment of the first embodiment. The detailed structures and actions of the air pump 4 are described as follows. Component parts and elements corresponding to those of the first embodiment are designated by identical numeral references, and detailed descriptions thereof are omitted. The air pump 4 is a piezoelectric air pump and includes a piezoelectric element 45, a resonance plate 46 and a gas inlet plate 47, which are stacked sequentially from top to bottom, and thus the gas inlet plate 47 faces the circuit board 1. The air pump 4 and the bottom plate 23 of the casing 2 are spaced apart from each other. The gas inlet plate 47 has at least one inlet aperture 471 for allowing the gas to flow in. The resonance plate 46 has a central aperture 461 in fluid communication with the at least one inlet aperture 471, and the resonance plate 46 is corresponding in position to the piezoelectric element 45. The resonance plate 46 has a movable part 462 surrounding the central aperture 461. The piezoelectric element 45 and the resonance element 46 are spaced apart from each other. The piezoelectric element 45 includes a vibration plate 451, at least one connection part 452, an outer frame 453 and a piezoelectric plate 454. The outer frame 453 is arranged around the vibration plate 451, and the at least one connection part 452 is connected between the outer frame 453 and the vibration plate 451 for elastically supporting the vibration plate 451. The surface of the vibration plate 451 is attached to the piezoelectric plate 454. As a voltage is applied to the piezoelectric plate 454, the piezoelectric plate 454 deforms owing to the piezoelectric effect. The piezoelectric plate 454 drives the vibration plate 451 to vibrate up and down. Meanwhile, the resonance plate 46 also vibrates up and down in resonance with the piezoelectric plate 454 owing to the Helmholtz resonance effect, thereby changing the pressure of the gas in the accommodation space 25. The gas is inhaled into the air pump 4 through the at least one inlet aperture 471 of the gas inlet plate 47, and the acetone sensor 3 adjacent to the first through hole 21 can measure the gas flowing in through the first through hole 21 and obtain the acetone concentration of the gas in real time. Then, the gas flows through the central aperture 461 of the resonance plate 46, and the gas flows to the two sides to be compressed in resonance with the movable part 462 of the resonance plate 46 and is discharged out through the vacant spaces among the at least one connection part 452 of the piezoelectric element 45. Finally, the gas is discharged out through the second through hole 22. The piezoelectric plate 454 drives the vibration plate 454 to vibrate up and down repeatedly so that the gas can be inhaled into the accommodation space 25 and discharged out from the accommodation space 25 into the environment so that the acetone sensor 3 can measure the acetone concentration of the gas continuously.

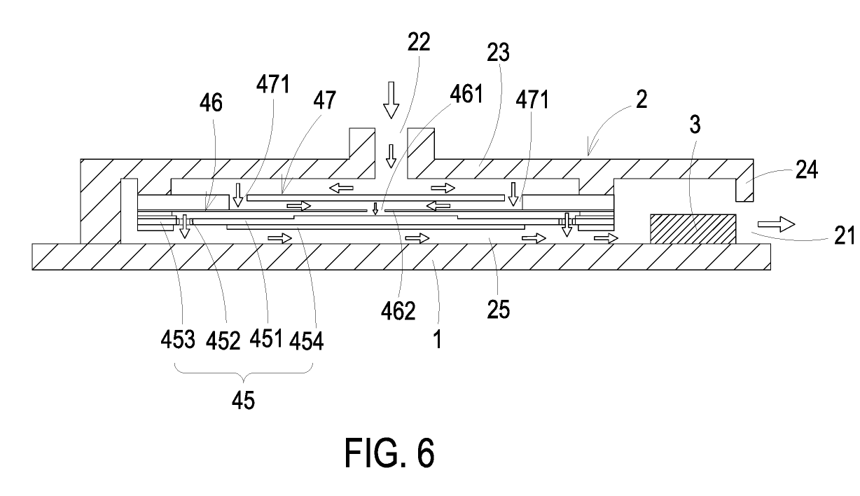

Please refer to FIG. 6, which is a schematic cross-sectional view illustrating the micro acetone detecting device according to a sixth embodiment of the present disclosure, wherein the arrow-shaped symbols indicate the direction for guiding the gas. The sixth embodiment is a derivative embodiment of the second embodiment. The detailed structures and actions of the air pump 4 are described as follows. Component parts and elements corresponding to those of the second embodiment are designated by identical numeral references, the structures of the air pump 4 is the same as that of the fifth embodiment, and detailed descriptions thereof are omitted. In this embodiment, the air pump 4 includes a gas inlet plate 47, a resonance plate 46 and a piezoelectric element 45, which are stacked sequentially from top to bottom and constructed on the casing 2. That is, regarding the assembly in the sixth embodiment, the gas inlet plate 47 faces the bottom plate 23 of the casing 2, and the air pump 4 and the bottom plate 23 of the casing 2 are spaced apart from each other. More specifically, the piezoelectric element 45 in the fifth embodiment faces the bottom plate 23 of the casing 2, but the piezoelectric element 45 in the sixth embodiment faces the bottom plate 23 of the casing 2. The direction of assembling the air pump 4 with the bottom plate 23 of the casing 2 in the sixth embodiment is reverse to the direction of assembling the air pump 4 with the bottom plate 23 of the casing 2 in the fifth embodiment. As a voltage is applied to the piezoelectric plate 454, the piezoelectric plate 454 deforms owing to the piezoelectric effect. The piezoelectric plate 454 drives the vibration plate 451 to vibrate up and down. Meanwhile, the resonance plate 46 also vibrates up and down in resonance with the piezoelectric plate 454 owing to the Helmholtz resonance effect so that the air pump 4 is enabled to transport the gas. The environmental gas is inhaled into the air pump 4 through the second through hole 22 and flows through the at least one inlet aperture 471 of the gas inlet plate 47 and the central aperture 461 of the resonance plate 46. Then, the gas flows to the two sides to be compressed in resonance with the movable part 462 of the resonance plate 46, and the gas flows into the accommodation space 25 through the vacant spaces among the at least one connection part 452 of the piezoelectric element 45. The gas flows through the acetone sensor 3, which is adjacent to the first through hole 21, so that the acetone sensor 3 can measure the inhaled gas and obtain the acetone concentration of the gas. Finally, the gas is discharged out through the first through hole 21. The piezoelectric plate 454 drives the vibration plate 451 to vibrate up and down repeatedly so that the gas can be inhaled into the accommodation space 25 to be compressed and discharged out from the accommodation space 25 into the environment, so that the acetone sensor 3 can measure the acetone concentration of the gas continuously.

From the above descriptions, the present disclosure provides a micro acetone detecting device, which utilizes the air pump to transport, collect and compress the gas to be provided to the acetone sensor, so that the acetone sensor can measure the acetone concentration of the gas and transmit the measured data to a controlling chip. The fat burning conditions of the user can be calculated according to the acetone concentration so that the blood glucose level of the user can be obtained for monitoring whether the blood glucose level of the user is too low. The gas transportation efficiency can be enhanced by using different air pumps so as to enhance the efficiency of detecting the acetone concentration. The user can exhale the gas to the micro acetone detecting device for obtaining the blood glucose concentration of the user. Consequently, a portable, convenient and rapid blood glucose detecting device can be provided to the user.

While the disclosure has been described in terms of what is presently considered to be the most practical and preferred embodiments, it is to be understood that the disclosure needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A micro acetone detecting device, comprising:
a circuit board;
a casing having a first through hole and a second through hole, wherein the casing is assembled on the circuit board, and an interior of the casing and the circuit board define an accommodation space;
an acetone sensor disposed in the accommodation space and electrically connected to the circuit board; and
an air pump disposed in the accommodation space and electrically connected to the circuit board,
wherein when the air pump is actuated to change pressure of gas in the accommodation space, the gas is transported to the acetone sensor so that the acetone sensor measures an acetone concentration of the gas in the accommodation space.

2. The micro acetone detecting device according to claim 1, wherein the acetone sensor is disposed in the accommodation space and disposed adjacent to the first through hole, wherein when the air pump is actuated to change the pressure of the gas in the accommodation space, the gas flows through the first through hole into the accommodation space, the gas is transported to the acetone sensor for allowing the acetone sensor to measure the acetone concentration of the gas in the accommodation space, and the gas is subsequently discharged out through the second through hole.

3. The micro acetone detecting device according to claim 1, wherein the acetone sensor is disposed in the accommodation space and disposed adjacent to the first through hole, wherein when the air pump is actuated to change the pressure of the gas in the accommodation space, the gas flows through the second through hole into the accommodation space, the gas is transported to the acetone sensor for allowing the acetone sensor to measure the acetone concentration of the gas in the accommodation space, and the gas is subsequently discharged out through the first through hole.

4. A micro acetone detecting device, comprising:
a circuit board;
a casing having a first through hole, a second through hole and a bottom plate, wherein the casing is assembled on the circuit board, the bottom plate and the circuit board define an accommodation space, and the accommodation space is in fluid communication with the first through hole and the second through hole;
an acetone sensor disposed in the accommodation space and electrically connected to the circuit board; and
an air pump disposed in the accommodation space and constructed on the bottom plate of the casing, wherein the air pump and the bottom plate of the casing are spaced apart from each other, the air pump is electrically connected to the circuit board, the air pump includes a nozzle plate and a piezoelectric assembly, and the nozzle plate has a central aperture, wherein in response to a voltage applied to the piezoelectric assembly, the piezoelectric assembly is actuated to drive the nozzle plate to vibrate so as to change the pressure of the gas in the accommodation space,
wherein when the air pump is actuated to change the pressure of the gas in the accommodation space, the gas is transported to the acetone sensor so that the acetone sensor measures an acetone concentration of the gas in the accommodation space.

5. The micro acetone detecting device according to claim 4, wherein the nozzle plate and the piezoelectric assembly of the air pump are stacked with each other and spaced apart from each other, and a frame is disposed between the nozzle plate and the piezoelectric assembly so that the nozzle plate and the piezoelectric assembly are spaced apart from each other.

6. The micro acetone detecting device according to claim 5, wherein the piezoelectric assembly, the frame and the nozzle plate of the air pump are stacked sequentially from bottom to top on the casing, and the nozzle plate faces and is spaced apart from the bottom plate of the casing, so that the air pump and the bottom plate of the casing are spaced apart from each other.

7. The micro acetone detecting device according to claim 6, wherein the acetone sensor is disposed in the accommodation space and disposed adjacent to the first through hole, wherein when the air pump is actuated to change the pressure of the gas in the accommodation space, the gas flows through the first through hole into the accommodation space, the gas is transported to the acetone sensor for allowing the acetone sensor to measure the acetone concentration of the gas in the accommodation space, and the gas is discharged out through the second through hole.

8. The micro acetone detecting device according to claim 5, wherein the nozzle plate, the frame and the piezoelectric assembly of the air pump are stacked sequentially from bottom to top on the casing, and the piezoelectric assembly faces and is spaced apart from the bottom plate of the casing, so that the air pump and the bottom plate of the casing are spaced apart from each other, wherein a resonance chamber is defined among the nozzle plate, the frame and the piezoelectric assembly.

9. The micro acetone detecting device according to claim 8, wherein the acetone sensor is disposed in the accommodation space and disposed adjacent to the first through hole, wherein when the air pump is actuated to change the pressure of the gas in the accommodation space, the gas flows through the second through hole into the accommodation space, the gas is transported to the acetone sensor for allowing the acetone sensor to measure the acetone concentration of the gas in the accommodation space, and the gas is discharged out through the first through hole.

10. The micro acetone detecting device according to claim 4, wherein the nozzle plate comprises a plurality of supporting parts, and the bottom plate of the casing has a plurality of fixing recesses, wherein the plurality of supporting parts are disposed and positioned in the plurality of fixing recesses of the bottom plate so that the nozzle plate and the bottom plate are spaced apart from each other.

11. The micro acetone detecting device according to claim 8, wherein the piezoelectric assembly comprises a piezoelectric plate, an auxiliary plate and a vibration plate, which are stacked sequentially from top to bottom, and the piezoelectric plate faces the bottom plate, wherein the auxiliary plate is disposed between the piezoelectric plate and the vibration plate, and severed as a buffer between the piezoelectric plate and the vibration plate for adjusting a vibration frequency of the vibration plate.

12. The micro acetone detecting device according to claim 11, wherein a thickness of the auxiliary plate is larger than a thickness of the vibration plate so as to adjust a vibration frequency of the piezoelectric assembly, wherein by controlling a vibration frequency of the gas in the resonance chamber to be close or the same as a vibration frequency of the nozzle plate, a resonance effect is generated between the resonance chamber and the nozzle plate for allowing the gas to be discharged out through the central aperture of the nozzle plate, thereby enhancing transportation efficiency.

13. A micro acetone detecting device, comprising:
a circuit board;
a casing having a first through hole, a second through hole and a bottom plate, wherein the casing is assembled on the circuit board, the bottom plate and the circuit board define an accommodation space, and the accommodation space is in fluid communication with the first through hole and the second through hole;
an acetone sensor disposed in the accommodation space and electrically connected to the circuit board; and
an air pump disposed in the accommodation space and constructed on the bottom plate of the casing, wherein the air pump and the bottom plate of the casing are spaced apart from each other, the air pump is electrically connected to the circuit board, and the air pump includes a gas inlet plate, a resonance plate and a piezoelectric element, wherein in response to a voltage applied to the piezoelectric element, the piezoelectric element is actuated to drive the resonance plate to vibrate so as to change pressure of gas in the accommodation space,
wherein when the air pump is actuated to change the pressure of the gas in the accommodation space, the gas is transported to the acetone sensor so that the acetone sensor measures an acetone concentration of the gas in the accommodation space.

14. The micro acetone detecting device according to claim 13, wherein the piezoelectric element, the resonance plate and the gas inlet plate of the air pump are stacked sequentially from top to bottom on the bottom plate of the casing, and the piezoelectric element faces and is spaced apart from the bottom plate, so that the air pump and the bottom plate of the casing are spaced apart from each other.

15. The micro acetone detecting device according to claim 14, wherein the acetone sensor is disposed in the accommodation space and disposed adjacent to the first through hole, wherein when the air pump is actuated to change the pressure of the gas in the accommodation space, the gas flows through the first through hole into the accommodation space, the gas is transported to the acetone sensor for allowing the acetone sensor to measure the acetone concentration of the gas in the accommodation space, and the gas is discharged out through the second through hole.

16. The micro acetone detecting device according to claim 13, wherein the gas inlet plate, the resonance plate and the piezoelectric element of the air pump are stacked sequentially from top to bottom on the bottom plate of the casing, and the gas inlet plate faces and is spaced apart from the bottom plate, so that the air pump and the bottom plate of the casing are spaced apart from each other.

17. The micro acetone detecting device according to claim 16, wherein the acetone sensor is disposed in the accommodation space and disposed adjacent to the first through hole, wherein when the air pump is actuated to change the pressure of the gas in the accommodation space, the gas flows through the second through hole into the accommodation space, the gas is transported to the acetone sensor for allowing the acetone sensor to measure the acetone concentration of the gas in the accommodation space, and the gas is discharged out through the first through hole.

18. The micro acetone detecting device according to claim 13, wherein the gas inlet plate of the air pump has at least one inlet aperture for allowing the gas to flow in, the resonance plate has a central aperture in fluid communication with the at least one inlet aperture, the resonance plate is aligned with the piezoelectric element and has a movable part surrounding the central aperture, and the piezoelectric element and the resonance plate are spaced apart from each other, wherein in response to a voltage applied to the piezoelectric plate, the piezoelectric plate is subjected to a deformation in piezoelectric effect so as to drive the vibration plate to vibrate up and down and change the pressure of the gas in the accommodation space, wherein the gas flows through the at least one inlet aperture of the gas inlet plate and the central aperture of the resonance plate sequentially, the gas flows to two sides to be compressed in resonance with the movable part of the resonance plate, and the gas is discharged out through the piezoelectric element.

19. The micro acetone detecting device according to claim 13, wherein the piezoelectric element of the air pump comprises a vibration plate, at least one connection part, an outer frame and a piezoelectric plate, the outer frame is arranged around the vibration plate, the at least one connection part is connected between the vibration plate and the outer frame for elastically supporting the vibration plate, and a surface of the vibration plate is attached to the piezoelectric plate.

20. A micro acetone detecting device, comprising:
at least one circuit board;
at least one casing having at least one first through hole and at least one second through hole, wherein the casing is assembled on the circuit board, and an interior of the casing and the circuit board define at least one accommodation space;
at least one acetone sensor disposed in the accommodation space and electrically connected to the circuit board; and
at least one air pump disposed in the accommodation space and electrically connected to the circuit board,
wherein when the air pump is actuated to change pressure of gas in the accommodation space, the gas is transported to the acetone sensor so that the acetone sensor measures an acetone concentration of the gas in the accommodation space.
